(19) United States
(12) Patent Application Publication
(10) Pub. No.: US 2009/0081264 A1
(43) Pub. Date: Mar. 26, 2009

(54) Cleansing article

(76) Inventor: Erez Zabari, Rehovot (IL)

Correspondence Address: AVIA KAFRI, 22 SHDEROT CHEN ST., TEL AVIV, 64166, IL

(21) Appl. No.: 12/289,993

(22) Filed: Nov. 10, 2008

Related U.S. Application Data
(63) Continuation-in-part of application No. 11/820,553, filed on Jun. 20, 2007.

(51) Int. Cl. A61K 8/00; A61K 8/97

(52) U.S. Cl. 424/401; 510/130; 424/725; 424/537

(57) ABSTRACT

A cleansing article which is a sponge impregnated with cosmetic soap, in which encapsulated beneficial agents are embedded and methods for its manufacturing are provided. The shell of a capsule according to one preferred embodiment of the present invention is made of materials soluble in water such as capsules typically utilized in the pharmaceutical industry. Alternatively a shell of a capsule may comprise the soap matrix and segments of the sponge. The beneficial agents considered include soothing materials, cosmetic agents, anhydrous self warming compositions, cooling agents, water in oil emulsions, foam generating compositions, natural fats, essential oils, waxes, crushed dried vegetables, crushed dried fruits, fruit extracts, honey, yogurt, vitamins, salts, minerals, cleansing solvents, salts from the Dead Sea, minerals from the Dead Sea and any combination thereof.

### REFERENCE TO PREVIOUS APPLICATIONS

The present application is a continuation-in-part of U.S. patent application Ser. No. 11/820,553, filed on Jun. 20, 2007, which is a section 371 filing of international patent application Ser. No. PCT/IL06/001449, filed on Dec. 12, 2006, claiming the benefit of priority from an application for patent No. IL172962, filed on Jan. 3, 2006.

### FIELD OF THE INVENTION

The present invention relates in general to cleansing products and in more particular to sponges impregnated with cosmetic soaps in which encapsulated and/or non capsulated skin beneficial agents and/or natural ingredients are embedded.

### BACKGROUND OF THE INVENTION

Disposable wet and/or dry wipes are common in the market place. Typically such wipes comprise a substrate made of non woven fibrous material impregnated with cleansing and/or antiseptic agents. Disposable wipes that may be additionally socked with one or more beneficial agents normally employed in cosmetic care treatments are commercially available.

Scrubbing sponges impregnated with cleansing and/or antiseptic agents are known. U.S. Pat. No. 4,866,806 discloses a surgical scrub sponge made of layered synthetic foam for cleanup prior to a surgical procedure. One layer serves to impart a degree of rigidity to the structure of the sponge; another layer having an abrasive surface provides for scrubbing the skin of a user. The intermediate layer is soaked with an antiseptic solution. The sponge immersed in the antiseptic solution is stored such that it is ready for an immediate use. In U.S. Pat. No. 4,291,697 another cleansing sponge providing for cleaning surfaces or a skin of a user prior to a medical treatment is disclosed. This sponge encloses a pressure rupturable pouch containing antiseptic solution. The antiseptic solution is dispensed off the pouch when the sponge is pressed against a surface or the skin of a user. Another sponge impregnated with cleansing agents for personal use is disclosed in U.S. Pat. No. 6,094,766. This sponge consists of two or three layers of synthetic foam at least one of which comprises impregnated cleansing agent. The first layer is made of a virgin foam sheet that is further impregnated with a combination of liquid hydrophilic foam and one or more cleaning agents all of which are being at a liquid phase. After the curing of this impregnated layer additional one or more layers of liquid foam either mixed with the cleansing agents as the aforementioned second layer or without such agents are laid one over the previous layer for a respective curing phase. The disclosed cleansing agents may include shampoo and/or a variety of other agents, such as a deodorant agent, a moisturizer agent, an anti-fungal agent, an anti-microbial agent, an anti-bacterial agent, and/or a fragrance agent. In world patent application WO07077552 a cosmetic soap including encapsulated beneficial agents embedded in a soap matrix is disclosed. The shell of a disclosed capsule is made of water soluble materials that are typically utilized in the pharmaceutical industry or from the soap matrix. The disclosed beneficial agents include soothing materials and/or cosmetic agents, such as cooling agents, anhydrous self warming compositions, water in oil emulsions, foam generating compositions, natural fats, essential oils, waxes, crashed dried vegetables, crashed dried fruits, fruit extracts, homey, yogurt, vitamins salts, minerals, minerals from the Dead Sea, fragrance dispensing materials, pigments and any combination thereof.

Any sponge integrally including dry cleansing agents or cosmetic soap, such as the cosmetic soap disclosed in WO07077552; that includes beneficial agents and natural ingredients among them; that provides a traveling user with the convenience of having a handy cleaning article that includes skin care agents that she or he are accustomed to, is beneficial.

### DETAILED DESCRIPTION OF THE PRESENT INVENTION

In accordance with the present invention a multi use cleansing article, which is a sponge impregnated with cosmetic soap, and a method for its manufacturing is provided.

The term sponge refers hereinafter to the substrate in which a cosmetic soap is impregnated. The substrate considered is an absorbent porous mass, made of natural fibrous, or synthetic fibrous and/or non fibrous, materials, such as typically used for washing.

Cosmetic soap refer hereinafter to any common cosmetic soap including but not limited to the cosmetic soap disclosed in world patent application WO07077552, the entire content of the specification of which is incorporated herein by reference. The disclosed soap consists of a soap matrix in which natural or artificially made beneficial agents are embedded. The beneficial agents are either encapsulated in capsules the shell of which is made of materials that are different from the soap matrix, or contained within capsules originated by cavities structured within the mass of the soap, the shells of which comprise the soap matrix.

A cleansing article of the present invention consists of a natural or artificially made sponge that is impregnated with a dry cosmetic soap.

The soap matrix is made in a cold or hot saponification process, followed by a curing phase, as known. The soap matrix in accordance with a preferred embodiment of the present invention comprises any of the following ingredients: sucrose, polyethylene glycol, glycerin, cocamidopropyl betaine, propylene glycol, and any combination thereof. Additionally, the soap matrix may include any of the following materials: sodium laureth sulfate, sodium stearate, sodium cocoate, sodium palmitate, ammonium lauryl sulfate, ammonium laureth sulfate, TEA lauryl sulfate, TEA laurate sulphate, isothionates based detergents, sulfosuccinate based detergents, sarcosinate based detergents, and any combination thereof.

A shell of a capsule in accordance with a preferred embodiment of the present invention is made of materials typically utilized for manufacturing capsules for the pharmaceutical industry, such as gelatin, lactose, cellulose, hydroxypropyl methylcellulose, ammonium methacrylate copolymer, ethylene/VA copolymer, acrylate copolymer; polybutylane terephthaleate, ceramide and any combination thereof.

The beneficial agents considered are soothing materials and/or cosmetic agents that clean, protect, moisturize, condition, warm and/or cool the skin after having been extracted from the capsules or cavities embedded in the cosmetic soap and applied to the skin. Preferable are known natural and/or artificially made materials typically employed for skin care treatments and/or utilized in creams for skin care, such as any of the following: natural fats including jojoba, soybean, sunflower, rice bran, avocado, almond, olive, sesame, lavender, wintergreen, peach kernel oil and castor. Also applicable are essential oils such as fish oils, menthol, jasmine, camphor, aloe vera, white cedar, bitter orange peel, cinnamon, bergamot, citrus calamus, pine, lavender, bay, clove, eucalyptus, lemon, thyme, peppermint, rose, sage, menthol, cineole, eugenol, citral, citronella, borneol, linalool, geraniol, evening primrose, camphor, thymol, spirantol, pinene, limonene and terpenoid oils. Waxes are applicable as well, for example carnauba, beeswax, lanolin and derivatives thereof. Other non classified ingredients are natural ingredients such as crushed dried vegetable and or fruits and or honey and fruit extracts such as aloe vera gel, or powdery extract such as papaya extract, mango extract, melon extract and vanilla planifolia extract, grape seed extract, ginseng extract, and yogurt. Vitamins and salts can also be used, such as vitamins A, B, C and E, and/or salts and minerals from the Dead Sea. Solvents typically used for cleansing such as polyethylene glycols, polypropylene glycols, isopropyl-palmitate, isopropyl myristate, mineral oils, silicon fluids, polysorbate and sorbitan fatty acids derivatives and high emulsifying systems can be used as well. Beneficial agents may also include anhydrous self-warming compositions such as dehydrated zeolite, or cooling agents such as menthyl lactate and any combination of essential oils typically used for cooling. Beneficial agents may also include creams consisting of water in oil emulsions typically used for skin care treatments. Beneficial agents may also include foam generating composition such as casein.

A procedure for manufacturing a cleansing article according to a preferred embodiment of the present invention is hereby described. First a predefined quantity of soap matrix of the cosmetic soap is prepared and stored in a first container at a temperature within the range of 50°-80° C. Such temperature range provides for retaining the soap at a liquid phase. A set of is molds are respectively lined each with a sheet of sponge, which is preferably made of polymeric foam such as polyurethane, or polyether-polyurethane at a predefined density and porousness. (Any other suitable foam materials which are commercially available having suitable densities and porosities are in accordance with the present invention.) Additionally, a predefined quantity of ready made capsules filled with a selected mixture of beneficial agents is stored in a second container. An exemplary capsule containing a given composition of beneficial agents has a shell made of gelatin, the volume of which is of a few cubic millimeters. An exemplary composition of beneficial agents includes powdery extracts of ginseng and yogurt, salt from the Dead Sea and casein. Then a predefined quantity of liquid soap from the first container is dispensed to form a layer enclosed within each of the lined molds. A given number of capsules taken from the second container are embedded in the layer of liquid soap prior to its solidification. At this stage each mold is covered with a sheet of the same sponge that lines its bottom and sidewalls. The rim of the covering sheet is coated with a suitable adhering material such that the cover is adhered to the lining sheet by pressing. A portion of the liquid layer is impregnated in the lining and the cover while the molds are cooled down to the room temperature. The molds are further kept at the same temperature for curing along a predefined time interval. By the end of the complete curing phase the sponges are taken off the molds and cut to fit with desired geometrical shapes. Optionally the molds lined with sheets of sponge are substituted by blocks made of sponge in which a central niche is structured. The liquid soap with or without the capsules are filled into the niche and a cover made of the same sponge is adhered to the rim of the respective base blocks.

A process according to another preferred embodiment of the present invention includes the following steps. First a set of molds each of which contain a piece of a natural or synthetic sponge are prepared. A predefined quantity of a liquid soap matrix is stored at a first container at a given temperature as described above. Another predefined quantity of a mixture of beneficial agents is stored in a second container at a temperature that is higher compared to the temperature of the first container. Then a predefined quantity of soap matrix is dispensed into each mold such that the liquid is impregnated within the contained sponges. Then a predefined quantity of beneficial agents dispensed off the second container is pressurized by means of an array of injectors into the liquid soap matrix impregnated within the sponge prior to its solidification. Preferably the apertures of the injectors are disposed at different heights relative to the bottom of the molds. By the end of such pressurizing a number of cavities encapsulating a portion of the mixture of beneficial agents, the walls of which comprise segments made of the soap matrix and/or the material from which the sponge is made are structured. At this stage a cover made of the same sponge is attached to the first sponge that lines the molds by means of a suitable adhering material. Then the filled molds are left for a predefined time interval for curing.

1. A cleansing article, wherein said cleansing article comprises a sponge and a soap matrix, said cleansing article comprising beneficial agents, wherein at least a portion of said beneficial agents is encapsulated in a plurality of capsules each having a shell, and wherein said cleansing article is manufactured by impregnating said soap matrix within said sponge.

2. A cleansing article as in claim 1, wherein said beneficial agents comprise any ingredient selected from a group of ingredients consisting of soothing materials, cosmetic agents, anhydrous self warming compositions, water in oil emulsions, foam generating compositions, and cooling agents.

3. A cleansing article as in claim 1, wherein said beneficial agents comprise any material selected from a group of materials including: natural fats, essential oils, waxes, crushed dried vegetables, crushed dried fruits, fruit extracts, honey, yogurt, vitamins, salts, minerals, cleansing solvents, salts from the Dead Sea, minerals from the Dead Sea and any combination thereof.

4. A cleansing article as in claim 1, wherein said shell comprises any compound selected from a group of compounds consisting of gelatin, lactose, cellulose, hydroxypropyl methylcellulose, ammonium methacrylate copolymer, ethylene/VA copolymer, acrylate copolymer; polybutylane terephthaleate, ceramide and any combination thereof.

5. A cleansing article as in claim 1, wherein said shell comprises said soap matrix.

6. A cleansing article as in claim 1, wherein said soap matrix comprises at least one material selected from a group of materials consisting of polyethylene glycol, propylene glycol, sodium laureth sulfate, sodium stearate, sodium cocoate, sodium palmitate, ammonium lauryl sulfate, ammonium laureth sulfate, TEA lauryl sulfate, TEA laurate sulphate, sucrose, polyethylene glycol, glycerin, cocamidopropyl betaine, isothionates based detergents, sulfosuccinate based detergents, sarcosinate based detergents, and any combination thereof.
